(19) United States
(12) Patent Application Publication
(10) Pub. No.: US 2015/0050387 A1
(43) Pub. Date: Feb. 19, 2015

(54) STARTER CULTURES FOR THE PRODUCTION OF FERMENTED MILK PRODUCTS

(71) Applicant: DMK Deutsches Milchkontor GmbH, Zeven (DE)

(72) Inventors: Marina Schomacker, Sittensen (DE); Michael Hahn, Heerlingen (DE)

(21) Appl. No.: 14/461,924

(22) Filed: Aug. 18, 2014

(30) Foreign Application Priority Data
Aug. 18, 2013 (EP) 13 180 794.3

(51) Int. Cl. A23C 9/123; C12N 1/20

(52) U.S. Cl. CPC A23C 9/1234; C12N 1/20; A23Y 2240/75; A23Y 2260/25; A23Y 2240/25; A23Y 2240/41; A23Y 2240/21; A23Y 2300/49; A23Y 2220/03
USPC 426/43; 435/252.4

(57) ABSTRACT

Suggested are starter cultures for the production of fermented milk products, containing
(a) a first mixture of five microorganisms comprising (a1) Streptococcus thermophilus, (a2) Leuconostoc species, (a3) Lactococcus lactis subsp. lactis biovar diacetylactis, (a4) Lactococcus lactis subsp. lactis und (a5) Lactococcus lactis subsp. cremoris, and
(b) a second mixture of three microorganisms comprising (b1) Streptococcus thermophilus, (b2) Lactococcus lactis subsp. lactis und (b3) Lactococcus lactis subsp. cremoris, as well as optionally
(c) a third group of microorganisms comprising (c1) Bifido bakterium lactis B12, (c2) Lactobazillus acidophilus, (c3) Leuconostoc cremoris or mixtures thereof.

### FIELD OF INVENTION

The invention is in the field of milk products and relates to starter cultures for producing fermented milk products, especially to a quark with enhanced taste, to a quark base mix containing these starter cultures, to a method for the production of quark base mix using these starter cultures as well as to the use of these microorganisms for the production of quark base mix.

### STATE OF THE ART

To produce quark, generally, skimmed milk is subjected to a temperature treatment and the proteins therein are denatured.

The subsequent addition of lactic acid bacteria and rennet performs what is termed coagulation (phase reversal) of milk. The casein coagulates and forms what is termed coagulum in the art. After ripening (8 to 20 h), the coagulum is agitated. The whey separation is initiated thereby, and the two phases are then separated in the separator. The liquid acid whey is processed in other ways and the quark base mix is adjusted to the desired fat and protein contents by adding cream.

However, these methods of the prior art have the disadvantage that the sensory assessment of the quark is assessed as unsatisfactory by many consumers. Either the product is faultless with respect to taste, but has a rough structure or, from the haptic impression, it is creamy, but leaves behind a slimy overall impression.

The complex object of the present invention was therefore to provide a quark mix having enhanced taste properties, which, on consumption, leaves behind directly a creamy impression, without the product tasting slimy.

### DESCRIPTION OF THE INVENTION

A first subject matter of the invention relates to starter cultures for producing fermented milk products, characterized in that they contain
(a) a first mixture of five microorganisms comprising (a1) Streptococcus thermophilus, (a2) Leuconostoc species, (a3) Lactococcus lactis subsp. lactis biovar diacetylactis, (a4) Lactococcus lactis subsp. lactis and (a5) Lactococcus lactis subsp. cremoris, and
(b) a second mixture of three microorganisms comprising (b1) Streptococcus thermophilus, (b2) Lactococcus lactis subsp. lactis and (b3) Lactococcus lactis subsp. Cremoris, as well as
   optionally
(c) a third group of microorganisms comprising (c1) Bifido bakterium lactis B12, (c2) Lactobazillus acidophilus, (c3) Leuconostoc cremoris or mixtures thereof.

Regarding to the microorganisms of the group (c), they are microorganisms, which usually are used as starter cultures for producing quark and are well described in the prior art. Using these starter cultures only products with the above mentioned unsatisfactory taste and sensory properties may be produced, so that the resulting quark base mix must be considerably further elaborated before they are ready-edible products to be launched into the mark.

After carrying out numerous tests with a wide variety of lactic acid bacteria the Applicant has identified a small group of microorganisms, which presents special advantageous properties.

Surprisingly, it has been found that the combination of the microorganisms (a) and (b) results in a clear improvement of the unsatisfactory taste and sensory properties of the quark base mix, so that ready-edible quark base mixes are immediately produced without the necessity of a sweet or Stretching post-treatment. These starter cultures mixtures are also compatible in a certain extent with the conventional starter cultures of the group (c).

### Starter Cultures

As explained relates the invention to starter cultures comprising at least two different mixtures of microorganisms, wherein the ingredients of the groups (a) and (b) overlap. The reason for this is that the starter cultures are not generally available as pure strains, but rather as mixtures. The starter culture mixture (a) alone would lead to an untypical taste-profile in the end product, namely a pronounced butter-like taste. Only the combination of the mixtures (a) and (b) leads to a product, which is perfect in terms of its taste.

Preferably, the starter cultures contain
(i) about 10 to about 90% by weight, preferably about 25 to about 75% by weight, and in particular about 40 to about 60% by weight of the mixture (a),
(ii) about 90 to about 10% by weight, preferably about 75 to about 25% by weight, and in particular about 60 to about 40% by weight of the mixture (b), and
(iii) 0 to about 20% by weight, preferably 0 to about 10% by weight, and in particular about 5 to about 10% by weight of the mixture (c),
   on condition that the amounts add to 100 wt.-%.

Particular preference is given to starter cultures which contain
(i) about 45 to about 60% by weight of the mixture (a) and
(ii) about 55 to about 45% by weight of the mixture (b)
   on condition that the amounts add to 100 wt.-%.

In a further preferred embodiment, the five microorganisms which form the mixture (a) and also the three microorganisms which form the mixture (b) are present in each case at about equal amounts. “About equal” in this context is taken to mean that in the mixture (a), the five microorganisms are each present in amounts of 20±5% by weight and in the mixture (b), the three microorganisms are each present in amounts of 33±5% by weight.

Instead of using the two commercially available preparations (a) and (b) together, it is, of course, in principle also possible to use the five microorganisms individually and then to mix them in such a manner that a starter culture mixture is obtained, with which the enhanced taste quark products are obtained. Such starter cultures then contain, preferably
(i) about 20 to about 30% by weight Streptococcus thermophilus,
(ii) about 5 to about 15% by weight Leuconostoc species,
(iii) about 5 to about 10% by weight Lactococcus lactis subsp. lactis biovar diacetylactis,
(iv) about 20 to about 30% by weight Lactococcus lactis subsp. lactis,
(v) about 20 to about 30% by weight Lactococcus lactis subsp. cremoris, and
(vi) 0 to about 15 wt.-% Bifido bakterium lactis B12,
on condition that the amounts add to 100 wt.-%.

Particular preference is given to starter cultures containing
(i) 25% by weight Streptococcus thermophilus,
(ii) 12% by weight Leuconostoc species,
(iii) 13% by weight Lactococcus lactis subsp. lactis biovar diacetylactis,
(iv) 25% by weight Lactococcus lactis subsp. lactis,
(v) 25% by weight Lactococcus lactis subsp. cremoris.

All stated microorganisms are freely available commercially.

### Quark Base Mix and its Preparation

Another subject matter of the invention relates to a quark base mix comprising the starter cultures of the invention.

Further the invention is directed towards a method for producing a quark base mix having enhanced taste properties, in which
(a) raw milk is subjected to a temperature treatment and the cream is separated off in such a manner that a non-acidified quark base mix is formed,
(b) the resultant mixture is subjected to a temperature treatment until denaturation occurs,
(c) the denatured product is admixed with starter cultures and rennet and optionally
(d) the quark base mix obtained after completion of fermentation is adjusted to a defined dry matter content and protein content.

Preferably, the quark base mix according to the invention has at 20° C. a Brookfield viscosity (RVT, spindle 1, 10 Upm) in the range from about 1000 to about 8000 mPas, preferably about 2000 to about 6000 mPas and in particular about 3000 to about 5000 mPas.

To produce the non-acidified quark base mix, solid components are initially separated off, and also the fat fraction of about 4% by weight is skimmed from the raw milk. This usually takes place in a special unit, preferably a separator. Such units are sufficiently known from the prior art. Separators from GEA Westfalia Separator GmbH are very widespread in the milk industry, and with which the two steps can be carried out individually or together.¹ Corresponding units are also described, for example, in DE 10036085 C1 (Westfalia) and are very well known to those skilled in the art, and thus for carrying out these method steps, no explanations are required, since they are considered part of general specialist knowledge. ¹(http://www.westfalia-separator.com/de/anwendungen/molkereitechnik/milch-molke.html).

The raw milk is preferably heat-treated in heat exchangers, wherein special plate heat exchangers have proved to be particularly suitable. There is a temperature gradient on the heat exchangers which, however, is selected in such a manner that the raw milk is heated for a residence time of at least 20 seconds and at most 60 seconds, preferably about 30 seconds, to a temperature of from about 70 to 80° C. and in particular about 72 to 74° C.

In the following step, the resultant non-acidified quark base mix is subjected to a thermal treatment. The denaturation then proceeding can proceed in a manner known per se, namely over a period of about 5 to about 10 min, and preferably about 6 min, and at temperatures of from about 85 to about 90° C., and in particular about 88° C.

The fermentation of the denatured preliminary product can also proceed according to the known methods of the prior art. For this purpose, suitable starter cultures and rennet add.

The temperature at which the fermentation takes place depends on the temperature range which is optimum for the microorganisms respectively used; typically, the temperature is in the range from about 18 to about 35° C., and preferably at about 30° C.

The quark base mix obtained after the fermentation and optionally after the stretching is then adjusted to the desired content of dry matter and proteins, for example by adding cream. Preferably, the dry matter content is about 15 to about 20% by weight, and in particular about 18% by weight. The protein content can be about 10 to about 15% by weight, and preferably about 12% by weight.

### EXAMPLES

### Examples 1 to 3, Comparative Examples C1 to C5

4 kg of skimmed milk were treated for 6 min at 88° C. so that all containing proteins were denaturized. The product was mixed with various starter cultures and rennet, maturated for about 18 h at 30° C. and subsequently stirred. Subsequently the fermentation product was given into a centrifuge and about 3.3 kg of acid whey was separated of as the liquid part. By adding cream the remaining quark mass (about 800 g) was adjusted to a dry mass of 18% b.w. and a protein content of 12% b.w. Subsequently the product was evaluated with respect to taste and sensory properties using a scale from 1 (=matches) to 6 (does not match at all) by a panel of experienced testers. The results are compiled in Table 1. Examples 1 to 3 are according to the invention, examples C1 to C5 serve for comparison. Presented are the average values of the evaluations.

TABLE 1
Taste and sensory properties of the quark base masses

| | | Taste | Sensory properties | | |
|---|---|---|---|---|---|
| Ex. | Starter Culture | bitter | creamy | smooth | slimy |
| C1 | Bifido bakterium lactis | 5.5 | 3.0 | 2.0 | 5.5 |
| | B12 | | | | |
| C2 | Lactobazillus acidophilus | 5.0 | 3.5 | 2.0 | 5.5 |
| C3 | Bifido bakterium lactis | 5.0 | 3.0 | 2.0 | 5.5 |
| | B12 + Lactobazillus acidophilus | | | | |
| | (1:1) | | | | |
| C4 | Mixture (a) | 3.0 | 2.5 | 2.0 | 4.0 |
| C5 | Mixture (b) | 4.0 | 2.0 | 2.0 | 4.0 |
| 1 | Mixture (a + b) = 75:25 | 2.0 | 4.0 | 3.5 | 2.0 |
| 2 | Mixture (a + b) = 50:50 | 1.5 | 4.5 | 4.0 | 1.0 |
| 3 | Mixture (a + b) = 25:75 | 2.5 | 4.0 | 3.5 | 1.5 |

The examples and the comparative examples clearly show, that the selection of starter cultures has a serious impact on the taste and sensory properties of the quark base mass.

Comparative examples C1 to C5 were conducted with usual lactic acid bacteria and mixtures (a) and (b) taken alone. The quark base masses exhibited a bitter taste and most of them showed a slimy consistence. In addition, the masses were little creamy and smooth.

By combining the mixture of starter cultures (a) and (b), however, quark base masses were directly obtained having a creamy and smooth appearance, without slimy consistence and little bitter taste.

What claimed is:

1. A starter culture for the production of fermented milk products, comprising
(a) a first mixture of five microorganisms comprising (a1) Streptococcus thermophilus, (a2) Leuconostoc species, (a3) Lactococcus lactis subsp. lactis biovar diacetylactis, (a4) Lactococcus lactis subsp. lactis und (a5) Lactococcus lactis subsp. cremoris, and
(b) a second mixture of three microorganisms comprising (b1) Streptococcus thermophilus, (b2) Lactococcus lactis subsp. lactis und (b3) Lactococcus lactis subsp. cremoris.

2. The starter culture of claim 1, comprising
(i) about 10 to about 90 wt.-% of the mixture (a),
(ii) about 90 to about 10 wt.-% of the mixture (b), and
(iii) 0 to about 20 wt.-% of a mixture (c) which is a third group of microorganisms comprising (c1) Bifido bakterium lactis B12, (c2) Lactobazillus acidophilus, (c3) Leuconostoc cremoris or mixtures thereof,
on condition that the amounts add to 100 wt.-%.

3. The starter culture of claim 1, comprising
(i) about 25 to about 75 wt.-% of the mixture (a),
(ii) about 75 to about 25 wt.-% of the mixture (b), and
(iii) 0 to about 10 wt.-% of a mixture (c) which is a third group of microorganisms comprising (c1) Bifido bakterium lactis B12, (c2) Lactobazillus acidophilus, (c3) Leuconostoc cremoris or mixtures thereof,
on condition that the amounts add to 100 wt.-%.

4. The starter culture of claim 1, comprising
(i) about 40 to about 60 wt.-% of the mixture (a),
(ii) about 60 to about 40 wt.-% of the mixture (b) and
(iii) 0 to about 10 wt.-% of a mixture (c) which is a third group of microorganisms comprising (c1) Bifido bakterium lactis B12, (c2) Lactobazillus acidophilus, (c3) Leuconostoc cremoris or mixtures thereof,
on condition that the amounts add to 100 wt.-%.

5. The starter culture of claim 1, comprising
(i) about 45 to about 55 wt.-% of the mixture (a), and
(ii) about 55 to about 45 wt.-% of the mixture (b),
on condition that the amounts add to 100 wt.-%.

6. The starter culture of claim 1, comprising the five microorganisms which form mixture (a) in about the same ratio.

7. The starter culture of claim 1, comprising the three microorganisms which form mixture (b) in about the same ratio.

8. The starter culture of claim 1, comprising
(i) about 20 to about 30 wt.-% Streptococcus thermophilus,
(ii) about 5 to about 15 wt.-% Leuconostoc species,
(iii) about 5 to about 10 wt.-% Lactococcus lactis subsp. lactis biovar diacetylactis,
(iv) about 20 to about 30 wt.-% Lactococcus lactis subsp. lactis,
(v) about 20 to about 30 wt.-% Lactococcus lactis subsp. cremoris, and
(vi) 0 to about 15 wt.-% Bifido bakterium lactis B12,
on condition that the amounts add to 100 wt.-%.

9. The starter culture of claim 1, comprising
(i) about 25 wt.-% Streptococcus thermophilus,
(ii) about 12 wt.-% Leuconostoc species,
(iii) about 13 wt.-% Lactococcus lactis subsp. lactis biovar diacetylactis,
(iv) about 25 wt.-% Lactococcus lactis subsp. lactis, and
(v) about 25 wt.-% Lactococcus lactis subsp. cremoris,
on condition that the amounts add to 100 wt.-%.

10. A quark base composition comprising the starter culture according to claim 1.

11. A method for the production of a quark base composition with improved taste characteristics, wherein
(a) raw milk is subjected to a temperature treatment and the cream is separated off in such a manner that a non-acidified quark base mix is formed,
(b) the resultant mixture is subjected to a temperature treatment until denaturation occurs,
(c) the denatured product is admixed with starter culture according to claim 1 and rennet and optionally,
(d) the quark base mix obtained after completion of fermentation is adjusted to a defined dry matter content and the protein content of the obtained quark base mix.

12. The method of claim 11, wherein the quark base composition is subjected to a temperature treatment at 85 to 90° C. over a time period from 5 to 10 min and is denatured thereby.

13. The method of claim 11, wherein the obtained denatured mass is mixed with the starter culture and rennet at about 18 to about 35° C.

14. The method of claim 11, wherein in the resultant quark base composition, the dry matter content is adjusted to about 15 to about 20 wt.-% and the protein content is adjusted to about 10 to about 15 wt.-%.

15. The starter culture of claim 1, additionally comprising a third group of microorganisms comprising (c1) Bifido bakterium lactis B12, (c2) Lactobazillus acidophilus, (c3) Leuconostoc cremoris or mixtures thereof.
